(19) United States
(12) Patent Application Publication
(10) Pub. No.: US 2018/0036053 A1
(43) Pub. Date: Feb. 8, 2018

(54) Dental / Prosthetic Implant

(71) Applicants: Elisandro R. Toscano, Orange, CA (US); Michael B. Frankel, Valley Village, CA (US); Yahya Mansour, Dallas, TX (US); Malek K, Mansour, Irvine, CA (US); Saam Zarrabi, Fort Worth, TX (US)

(72) Inventors: Elisandro R. Toscano, Orange, CA (US); Michael B. Frankel, Valley Village, CA (US); Yahya Mansour, Dallas, TX (US); Malek K, Mansour, Irvine, CA (US); Saam Zarrabi, Fort Worth, TX (US)

(21) Appl. No.: 15/668,329

(22) Filed: Aug. 3, 2017

Related U.S. Application Data
(60) Provisional application No. 62/370,533, filed on Aug. 3, 2016.

(51) Int. Cl. A61B 17/86; A61F 2/44; A61F 2/46; A61B 5/00; A61F 2/72; A61B 5/11; A61C 8/00; A61B 5/04

(52) U.S. Cl. CPC A61B 17/869; A61C 8/0039; A61C 8/0074; A61C 8/0068; A61C 8/0012; A61C 8/0053; A61C 8/008; A61F 2/4455; A61B 5/04001; A61B 5/686; A61F 2/72; A61B 5/1112; A61F 2/468; A61F 2002/3085

(57) ABSTRACT

Improvements in a dental/prosthetic implant is disclosed. The implant includes interior and exterior threaded surfaces. The use of both interior and exterior expanding threaded surfaces for integrations of the insert and a prosthetic with the same implant. The implant is immediately usable under load and promotes rapid integration with bone growth. The expanded insert essentially makes contact with the tapped bone surfaces where loads can be immediately applied so a person can utilize the prosthetic implant. The implant can further include security devices GPS, ID with medical records making removal of the implant difficult to extract. A cushioning member may be further integrated. The implant/abutment can include a surface with a plurality of contacts with sufficient gold contact points to attach residual nerve endings, during implanting surgery to provide nerve identification, send, receive, target so as to exploit proprioceptive memory or retraining.

### CROSS REFERENCE TO RELATED APPLICATIONS

This application is a continuation-in-part of applicant's co-pending application Ser. No. 62/370,533 filed Aug. 3, 2017 the entire contents of which is hereby expressly incorporated by reference herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

This invention relates to improvements in a dental or prosthetic implant. More particularly, the present dental or prosthetic implant is a wire thread insert device that is anchored into a bone and a tooth or prosthetic device is secured using the wire thread insert device. Electrical contacts can also be used with a prosthetic to connect nerves to the prosthetic to operate sensors and servos or mechanisms.

### Description of Related Art Including Information Disclosed Under 37 CFR 1.97 and 1.98

Dental implant complications, are generally related to poor planning, improper diagnosis, or inappropriate treatment option. The most common complications are inflammation or infection of the implant, bone or gum tissue loss, aesthetic issues, and functional problems.

A dental implant may heal poorly and lose integration due to several reasons including, but not limited to; inflammation or infection following surgery, poor bone quality, inadequate amount of bone, micro-motion from loading the implant too early, poor surgical technique during placement, poor stability during placement, over preparation of the site, traumatic surgery with poor handling of bone and gum tissue, smoking and poor oral hygiene.

For people with bad teeth, the only solution is with a dental implant this is accomplished by inserting or bonding an anchor into the bone and then implanting a dental appliance onto the insert. Various devices and methods have been utilized to anchor the implant into the bone. A number of these require a period of time between insertion of the implant to allows the bone to grow around the implant. Prior art includes an external thread which is driven into the bone and an internal thread which accepts an insertion tool.

For prosthetic implants such as, but not limited to amputated appendages, hip replacement or other bone/joint replacements, the bone is cut off and an implant is inserted into the bone. It often takes time for the bone to grow around the insert. In the case of an amputee, the appendage is missing, but the nervous system in intact in the remaining portion of the body. The nervous system includes connections for muscle control and touch/feel receptors. Connection from the disconnected nervous system to a prosthetic will allows the amputee to operate and feel objects using the same nervous system connections that originally existed.

A number of patents and or publications have been made to address these issues. Exemplary examples of patents and or publication that try to address this/these problem(s) are identified and discussed below.

U.S. Pat. No. 4,960,381 issued to Gerald A. Niznick on Oct. 2, 1990 discloses a Screw-Type Dental Implant Anchor. The dental implant anchor includes an externally-threaded body portion having internal structure for engaging an insertion tool. The body portion can be joined to a top portion having an unthreaded exterior wall. This internal is inside a top portion or inside the body portion of the anchor. While this type of dental implant is insertable into a bone, the anchor has a fixed outside diameter and requires bone grown to anchor the implant.

U.S. Pat. No. 6,464,500 issued to Don D. Popovic on Oct. 15, 2002 discloses a Dental Implant and Abutment System. The system uses a dental implant anchor that includes an external body surface that can have threads or be non-threaded and a uniquely designed internal portion for engaging screws, abutments, and insertion tools in one step. The system uses tapered threads and one-way barbs to temporarily secure the implant into the bone while the bone grows around the implant. This type of dental implant also has a fixed outside diameter and requires bone grown to anchor the implant.

U.S. Pat. No. 7,059,855 issued to Albert Zickman et al., on Jun. 13, 2006 discloses a Dental Implant System. The system dental implant system includes a dental implant having an external thread for threading into bone and an internal thread for attachment of a prosthesis. The internal thread has a plurality of notches which accept a complimentary shaped insertion tool.

U.S. Pat. No. 5,171,324 issued to Donna L. Campana on Dec. 15, 1992 discloses a Prosthetic Hip Stem Implant with Torque Adapter. The prosthetic hip stem implant and torque wrench adapter. The hip stem includes an extraction bore formed through its body and a plurality of recessions formed in the body upper surface. A lip extends outwardly of the body and including alignment indicia for visually aligning the implant during seating. While this prosthetic operates as a bone/joint replacement the shaft is smooth and requires bonding and/or bone growth to bond to the bone.

What is needed is a dental/prosthetic implant that self-expands into the bone to secure the implant upon installation. The implant should further include internal threads for engaging a prosthetic. The prosthetic should also include contacts for connecting to nerves to allow for an articulatable prosthetic. The proposed dental/prosthetic implant provides the solution.

### BRIEF SUMMARY OF THE INVENTION

It is an object of the dental/prosthetic implant to include interior and exterior threaded surfaces. The use of both interior and exterior threaded surfaces allows for both integrations of the wire thread insert and a prosthetic with the same implant. The wire thread insert is also found under the registered names of Helicoil, Spiralock or similar threaded insert can be coiled into a hole in a semi-coiled configuration. Once the insert is threaded into the bone, the insert can expand into the hole.

The 60-year proven efficacy of a Helicoil's fit when used as a dental implant during the healing process can ameliorate the propensity for standard implants to fail under these conditions or effect. The reason for this is a Helicoil provides superior disbursed loading, avoiding micro motion, promoting adhesion and stability. Any normal solid threaded implant, like all screws have around 20% adhesion, hence poor stability. Any motion and or poor fit can lead to failure during the healing process. The consequences can be severe to create or restore a positive outcome. It is well established that Helicoil adhesion is 70% or better and loading and stability as well are commensurate with that advantage. When high precision Helicoil implants are used these vectors can approach 90% efficacy.

It is an object of the dental/prosthetic implant for the implant to be immediately usable under load. The expanded insert essentially makes contact with the tapped bone surfaces where loads can be immediately applied so a person can utilize the prosthetic implant.

It is an object of the dental/prosthetic implant to be fabricated from a material that allows rapid integration with bone growth. Surface treatment or plating can be imparted into or onto the insert. Some materials or plating improve and/or accelerate bone growth, modeling and remodeling.

It is another object of the dental/prosthetic implant to operate with human and/or animal bone. The implant is not restricted to humans and can be equally inserted into animals to replace a damaged/missing limb, appendage or tusk. The implant allows nearly full mobility to return where an animal can be returned to service or returned to its prior location or habitat.

It is another object of the dental/prosthetic implant to include security devices GPS, ID with medical records, miniature transceivers in hi risk and targeted individuals as well as military hostile environment field work could be more securely attached to bone, Difficult to detect extract.

It is another object of the dental/prosthetic implant to include a cushioning member as an interface between abutment and the end of a wire thread insert to compresses without extruding to absorb pressure, shock. The cushion would mimic the shock absorbing properties of periodontal ligaments. Their efficacy in absorbing shock in leg prosthetics, walking running climbing is obvious.

It is still another object of the dental/prosthetic implant to form a base to attach a prosthetic on an amputee. The implant would have both Osseo integrated wire thread insert surrounding a percutaneous tapered implant, and external hollow shaft with and internal wire thread insert to which an abutment can be attached capable of supporting multiple interfaces. The multiple interfaces can be changed at upgraded as technology and need changes to provide optimal benefit the amputee. The internal/external abutment which allows for surrounding residual muscle to be attached and used to activate mechanical prosthetic arms, hands or feet.

It is still another object of the dental/prosthetic implant to include a surface with a plurality of contacts with sufficient gold contact points to attach residual nerve endings, during implanting surgery to provide nerve identification, send, receive, muscle control, fatigue, temperature, angle, touch so as to exploit proprioceptive memory or retraining.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1A shows a first perspective view of a jaw bone with a dental implant.

FIG. 1B shows a second perspective view of a jaw bone with dental implants.

FIG. 2A shows an exploded diagram of small incisors that are adhered to the abutment.

FIG. 2B show an exploded diagram of large molars with screws through the tooth.

FIG. 3 shows an assembled view of a bisected large molar with the wire threaded insert.

FIG. 4 shows various abutment attachments.

FIG. 5A shows a common osseointegrated implant.

FIG. 5B shows a wire thread osseointegrated implant.

FIG. 6 shows an X-Ray of an osseointegrated implant.

FIG. 7 shows a spine with fusion surgery.

FIG. 8 shows an X-Ray of an implant through a spinal vertebrae.

FIG. 9 shows an Electromyogram.

FIG. 10 shows a nerve stimulator and an electromyographic (EMG) monitor.

FIG. 11 shows a block diagram of the nervous system detection of pain.

FIG. 12 shows a graphical representation of the nerve sensory detection.

FIG. 13 shows a robotic interface from a sensory and muscle interface.

### DETAILED DESCRIPTION OF THE INVENTION

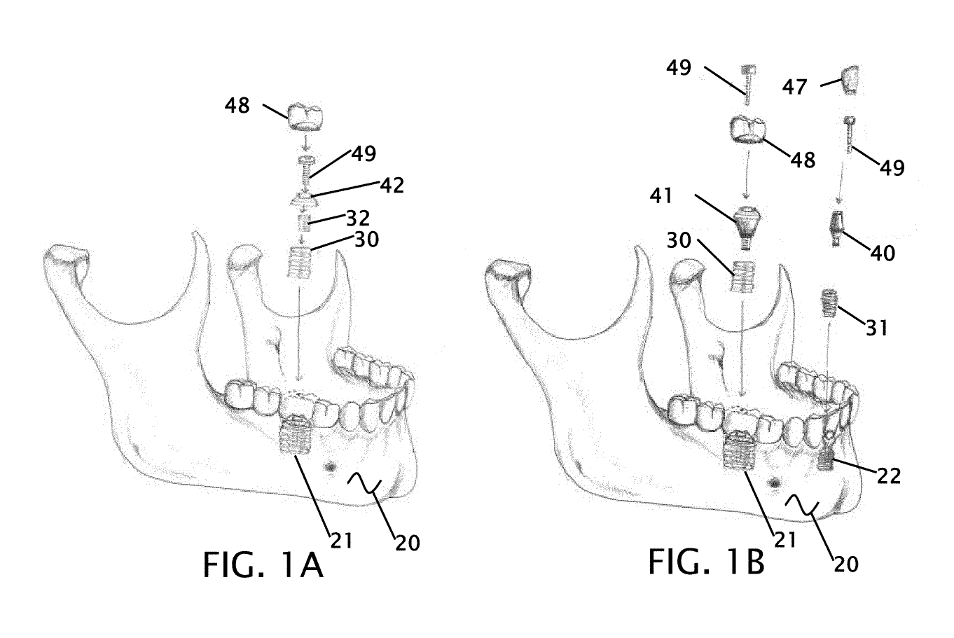

FIG. 1A shows a first perspective view of a jaw bone 20 with a dental implant and FIG. 1B shows a second perspective view of a jaw bone 20 with dental implants. The dental implant in these figures is a wire threaded insert 30. These wire threaded inserts 30 are also known as registered trademarks as Helicoils, Twinserts, Tanged, & Tangless and others. The wire threaded inserts are for use in allopathic medicine with a primary application in a superior kind of dental implant(s) in human and veterinary medicine. and wherever attachment to bone of any material is indicated. Medical training, fossil assembly, art, jewelry, etc. The wire threaded insert 30, 31 is a proven device for superior threaded attachment/connector function since 1938 and as per the wire threaded insert 30, 31 a suggested allopathic remedy (i.e. dental implants) would also prove superior because of the proven technical advantages as well as vastly improving current dental implant technology.

It's established and proposed advantages are numerous including but not limited to wire threaded inserts are stronger and longer lasting especially when mating materials of different media. A mechanically tapped hole 21/22 in general, will exhibit a surface roughness eight times more than the surface of a wire threaded insert 30, 31 which will overcome surface contact disparity via very tight tolerances and increased clamping action including self-adjustment when receiver medium (i.e. bone) deteriorates.

A medical grade titanium wire threaded insert dental implant is mostly inert, anti-corrosive, anti-magnetic, withstands extremes of pressure, temperature, acidity, and stress as is typical of wire threaded insert behavior in general. Such conditions are resident in the human/animal mouth. Bolt failure and thread stripping is reduced due to torque, resident and progressive pitch angle errors, pressure, stress and movement.

Typical thread clamping is up to 70% at the two threads at the collar leaving the tolerances fit highly diminished over the majority of the threads length. Pitch errors and rough contact greatly exacerbates the problem which continues to increase due to said forces, of stress, pressure, movement, temperature variance, material deterioration (i.e. bone). wire threaded insert 30, 31 stretch compensates for these variables and distributes loading and clamping more evenly even when progressive pitch error occurs which can be expected in bone. Radial and axial elasticity actually allows the shearing load threshold (and bolt failure) to be converted to advantage by transformation to an advantageous radial load fit (hoop stress) evenly distributing the loading over the entire length of the threads. Bolt breakage, cracking, thread shearing is virtually eliminated though bone tissue in the mouth can be expected to exert conditions which become problematic in a typical dental implant causing the wire threaded insert 30, 31 to respond so as to improve the fit.

Medical grade titanium is known to osseo integrate further enhancing improved and self-adjusting fit of a dental implant using said material achieving tremendously improved integration via all the aforementioned advantages of properly engineered wire threaded insert 30, 31. This is an extraordinary phenomenon in which intrinsic problems with current dental implant can be overcome via wire threaded insert 30, 31 advantages and osseo integration in combination.

Metal medical devices which reside in the body must be inert and not react to conditions in the body. Metal grade titanium alloys are the metal of choice. Developments in metallurgy seek to improve the inert characteristics of titanium via nanostructuring eliminating possible aged material leaking of toxic alloyed metals. Under certain diagnostic conditions a thin coating of zirconium oxide could be applied to the wire threaded insert 30, 31 in a specialized wire threaded insert 30, 31 where alloyed metals leakage might be a concern, environmental sensitivities, allergies, compromised immune systems, etc. The use of nanotechnology to alter granularity at nanoscale specifically to produce inert medical grade titanium wire thread insert excluding toxic alloys without and improving behavior characteristics and weight. In some cases, bone to metal osseointegration of implants between bone and metal is with a glue with a thickness of about a molecule. In a sense, a wire threaded insert 30, 31 is a storehouse of potential energy as expressed as function of a linear spring as described in the below equation:

U(x)−½kx²

Measured as an SI (derived energy potential) micro joule (the millijoule (mJ) is equal to one thousandth (10−3) of a joule.) It is this potential energy which makes the wire threaded insert 30, 31 dental implant a dynamic self-adjusting, self-correcting, self-integrating, semi-permanent prosthetic device as to opposed to a standard dental implant which is a static prosthetic and in a given difficult environment (the mouth) problematic conditions arise or given enough time (life span of device) will itself become problematic and or fail necessitating replacement. Such quantitative measurements when related to the diagnostic properties of healthy and compromised bone will determine the spring strength to be chosen when choosing and implementing a wire threaded insert 30, 31 dental implant as an allopathic prosthetic virtually establishing a highly tuned relationship between the implant and the bone.

Where bone 20 is severely compromised or too thin walled a threaded bushing can be used instead of a wire threaded insert 30, 31 which would give strength and support but only when the bone mass is insufficient to receive the tiniest wire threaded insert 30, 31 which remains the preferred choice as bushings retain the same problems as a standard implant, though function as a prosthetic at least.

In these figures, the tooth or teeth are extracted. A hole is drilled to the minor diameter for the wire threaded insert 30, 31 to a desired depth. The drilled hole is tapped using a bottom tap. The wire threaded insert 30, 31 is threaded into the tapped hole 21/22. If a tang is present in the wire threaded insert 30, 31, the driving tang is broken off or otherwise removed. The wire threaded insert 30, 31 is installed to a depth within the jaw bone 20.

In FIG. 1A an inner wire threaded insert 32 is then installed. An abutment 40, 41 or 42 is then installed into the wire threaded insert. If the tooth is a molar 48 then a screw 49 is generally placed through the molar implant 48 and the head of the screw is then covered with a filling. If the tooth is an incisor 47 then the screw 49 is threaded into the abutment and the implant 47 is bonded to the screw 49. While the figure shows the implant in the jaw bone, it can also be installed in the upper jaw bone.

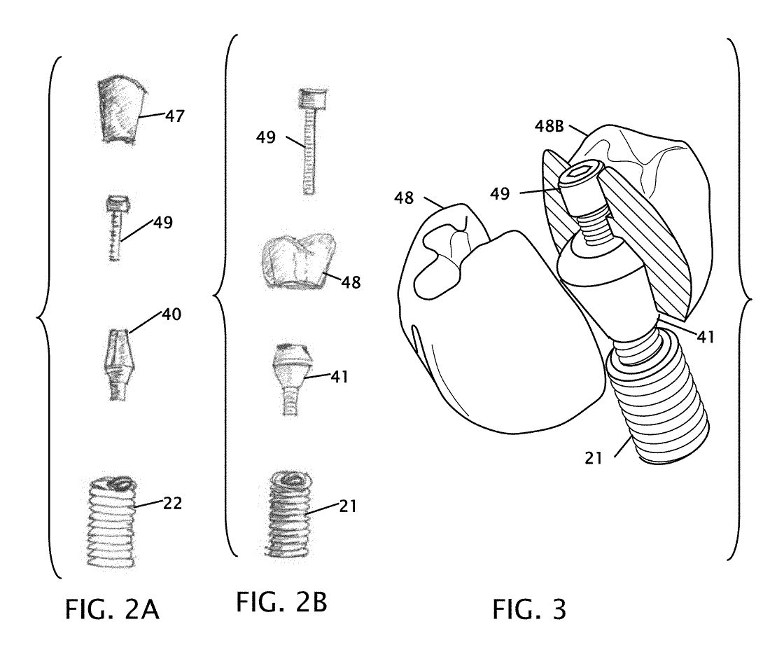

FIG. 2A shows an exploded diagram of small incisors that are adhered to the abutment, FIG. 2B show an exploded diagram of large molars with screws through the tooth and FIG. 3 Shows an assembled view of a bisected large molar with the wire threaded insert. These embodiments are used with tangles free-running inserts, tangles screw-locking inserts, tanged free-running inserts and tanged screw-locking inserts 21/22. An abutment 40, 41 is installed into the wire threaded insert 21/22. If the tooth is a molar 48, 48A/48B then a screw 49 is generally placed through the molar implant 48, 48A/48B and the head of the screw is then covered with a filling. If the tooth is an incisor 47 then the screw 49 is threaded into the abutment and the implant 47 is bonded to the screw 49. While the wire threaded insert may be shown straight (not tapered) it is contemplated that the wire threaded insert can be tapered to be narrower at the base. The wire threaded insert is essentially square in cross-section, but could have rounded edges to match the tap that is used to tape the bone.

Wire threaded inserts such as Spirallock have a wedge ramp design that has been produced in wire thread inserts to offer the same vibration resistance and reusability while bringing higher strength and clamp load capability to titanium. The wire thread inserts are available in two styles: tanged and Drive Notch engineered with no tab. They are particularly effective in application for aerospace, electronics and medical industries.

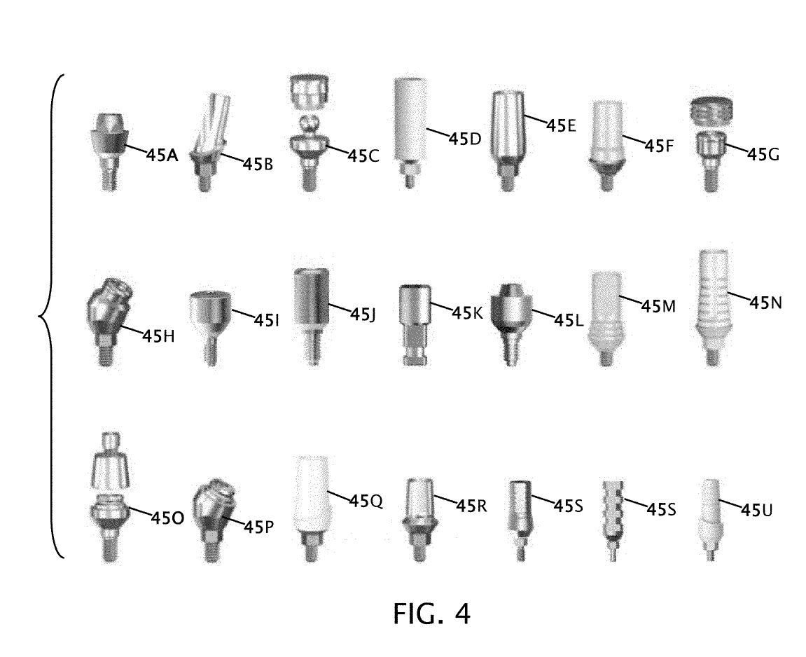

FIG. 4 shows various abutment attachments. These abutments are for dental applications, but it is further contemplated that the abutments can have equivalent applications for prosthetic devices for limbs. The abutments in this figure include, but are not limited to aesthetic abutment 45A, angled contoured 45B, ball attachment abutment 45C, casting abutment 45D, full contour abutment 45E, gold/plastic castable abutment 45F, Global Positioning Satellite (GPS) abutment 45G, 15 and 30 degree GPS attachment abutment 45H, healing abutment 45I, locator abutment 45j, lab analogs abutment 45K, multi-unit abutment 45L, plastic non-engaging castable abutment 45M, plastic temporary abutment 45N, screw receiving abutment 45O, 15 and 30 degree screw receiving abutment 45P, straight contoured Zirconia/Titanium abutment 45Q, straight snap-on abutment 45R, Titanium abutment 45S, transfer abutment 45T and Zirconia abutment 45U. These abutments may further include a GLYD ring for compression absorption.

Numerous security devices GPS, RFID, ID with medical records, miniature transceivers in hi risk and targeted individuals as well as military hostile environment field work could be more securely attached to bone, Difficult to detect extract.

Implant technology has advanced significantly over the past years, there are ongoing issues with the loosening and fracture of implant screws. The load on the back teeth has been shown to be 50 to 80 kilograms, particularly for those who habitually grind their teeth. This application of high loads over prolonged periods has led to the failure of the implant screws, so improved fracture resistance would provide significant benefits for people who sometimes struggle with the maintenance of their implants.

Resorbable Implants

There is huge interest in biodegradable or bio-resorbable implants that gradually dissolve during the healing process, reducing the risks of inflammation and eliminating the need for repeat surgeries to replace or remove implants. Magnesium is a prime candidate for such resorbable implants as it is entirely biocompatible—and many people actually have a magnesium deficiency. Magnesium can dissolve too fast.

Research is assessing the advances that can be achieved in a range of metals when subjected to this process—some anticipated, such as increased strength, others unexpected, such as greater corrosion resistance and increased biocompatibility.

Metals are actually made of small crystallites, or grains. Applying mechanical load to deform the metal in specially designed processes breaks these into smaller and smaller fragments, down to a nanoscale granularity, while maintaining the material's overall structure. The more times the metal is pushed through the die, the smaller the grains become. The smaller the grains, the stronger the material, although there is a natural limit to both the reduction in grain size and the improved strength.

Titanium screws fixed into the jaw to hold artificial teeth, which have become a popular alternative to dentures. The screws are currently made of a titanium alloy that includes aluminium and vanadium to provide additional strength, but both elements are considered by some to be potentially toxic. Pure titanium is more biocompatible, but it doesn't have the strength of the alloy. However, we can take commercially pure titanium and use nanostructuring to give the material o extra strength. This leaner, cleaner and stronger titanium compensates for the loss of the alloying elements.

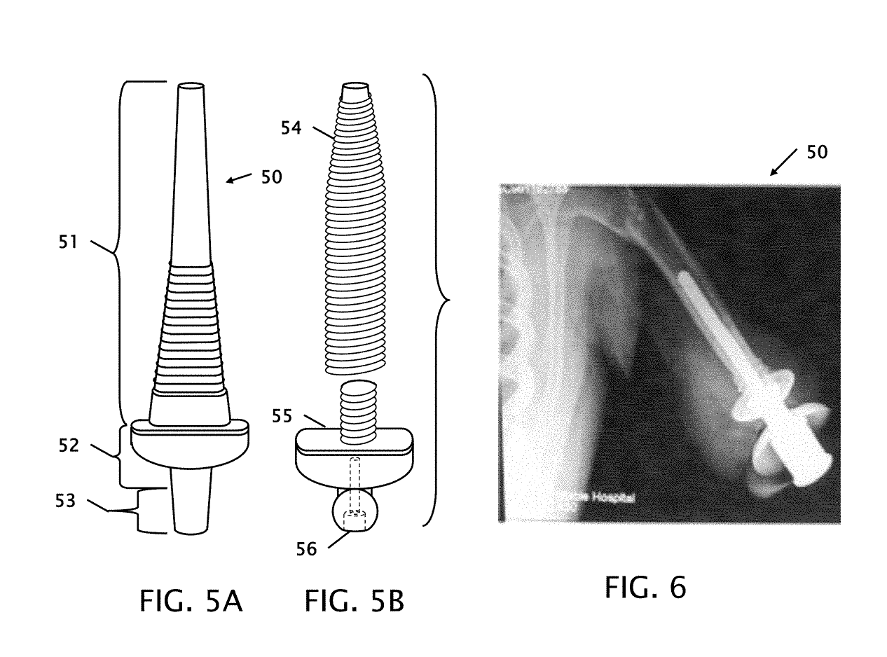

FIG. 5A shows a common osseointegrated implant 50. The osseointegrated implant has a number of components of an osseointereated implant 51 that is inserted onto a bone. The hilt creates a skin /implant interface 52. The end of the skin/implant interface extends through the skin of the person or animal. The end of the osseointegrated implant 50 is a percutaneous implant 53.

FIG. 5B shows a wire thread osseointegrated implant. This implant can be scaled to accommodate the size of the bone from a leg bone to a finger or thumb. The osseointegrated implant has a wire threaded insert 54. This insert 54 shows a tapered end that is threaded into a tapped hole in a bone. The abutment 55 threads into the wire threaded insert 54. In this embodiment, the end of the abutment 55 has a ball. A threaded fastener 56 secures a prosthesis into or onto the ball.

FIG. 6 shows an X-Ray of an osseointegrated implant 50 on an arm where the arm has been severed above the elbow. There are a number of problems with socket-suspended prostheses. Most patients report a range of problems with the prosthetic socket. By surgically implanting a titanium screw into the residual bone, the prosthesis can instead be attached using a socket. The prosthesis always fits, always attaches correctly and is always held firmly in place. Osseo integrated prostheses for the rehabilitation of amputees enhances the quality of life and offers a greater degree of freedom in everyday life. The advantages include:

A stable attachment for bone-anchored prosthesis is attached without using a socket, thereby ensuring stability. This also allows for the benefit of requiring a minimal time to attach the prosthesis.

amputation, but osseointegration is currently the best cosmetic option. Other alternatives are toe-to-finger transfer or the surgical creation of a thumb using the index finger.

Implant Surgery

The treatment consists of two operations with a three- to four-month interval. In the first operation, a specially constructed titanium screw (fixture) is installed in the residual bone. The period of hospitalisation is usually about two to four days.

In the second operation, an abutment is added to the fixture. The abutment protrudes through the skin. The period of hospitalisation is approximately two to four days and you will only be able to undertake limited exercise according to your training programme in the following weeks, thereby allowing the skin to heal.

Rehabilitation

When it comes to above-elbow amputation, loading of the bone can start (using a short training prosthesis) after the skin penetration area has healed, which is approximately three to six weeks after the second operation.

Everyday exercise is based on loads on the prosthesis on a standard set of scales. By gradually increasing the load, the strength of the bone will improve. Approximately twelve weeks after the second operation, a prosthesis can be fitted. In the case of below-elbow and thumb amputees, the movement of adjacent joints is exercised until the prosthesis is fitted.

There is normally a four- to six-month interval between stages one and two for transhumeral and transradial patients and four months for thumb patients. In a few selected cases where there is good bone quality, stages one and two have been performed simultaneously.

When it comes to transhumeral patients, a short training prosthesis is used three to six weeks after stage two, with increasing weights and loading until the patient reaches the weight of the final prosthesis. It could be a myo-clectric, bodypowered or cosmetic prosthesis. No short training prosthesis is used for transradial or thumb patients.

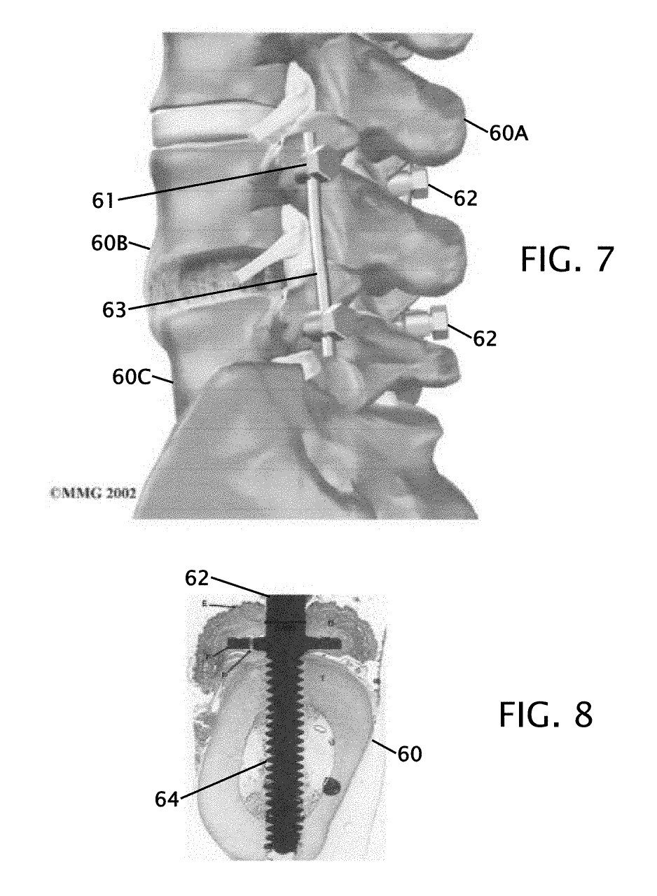

FIG. 7 shows a spine vertebrae with fusion surgery. This fusion surgery joins one or more bones 60A, 60B, 60C into one solid bone. In this case 60B and 60C. This keeps the bones and joints from moving. In this procedure, the surgeon lays small grafts of bone over the back of the spine. Most surgeons also apply metal plates or rods 63 and screws 61, 62 to prevent the two spinal vertebrae 60B, 60C from moving. This protects the graft so it heals better and faster.

FIG. 8 shows an X-Ray of an implant through a spinal vertebrae. The screw or wire threaded insert 64 is threaded into the vertebra 60. The threaded insert 64 is screwed from the head 62 of the screw or abutment.

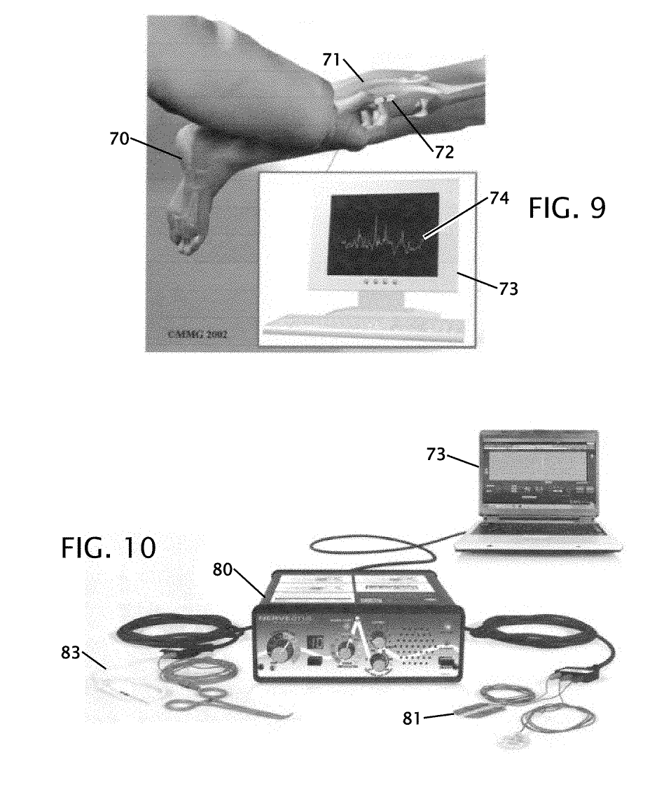

FIG. 9 shows an electromyogram. In the case of limb replacement on an abutment, the body has still lost the ability to control the lost limb or digit. Electromyogram allows for sampling data from nerves 72 that control muscles 71 to control a foot 70 or other body part. The signal to/from the nerves 72 can be detected and shows on a computer 73 display 74. The nerves can be monitored and can be used to create a replacement limb, hand or digit.

FIG. 10 shows a nerve stimulator and an electromyographic (EMG) monitor. This device combines a nerve simulator 81 and an electromyographic (EMG) monitor into an integrated surgical tool. A surgical instrument is enhanced to become a monopolar probe 83 the continuously applies a stimulation pulse to soft tissue while the EMG monitor detects, interprets and records muscle response evoked by stimulation. Once an evoked EMG is identified. The device 80 produces an audio alarm allowing a surgeon to maintain attention on the surgical field. A computer can also be connected to display 73 the signal. This device 80 significantly reduces nerve location time while improving patient safety and decreasing surgeon stress during difficult dissections.

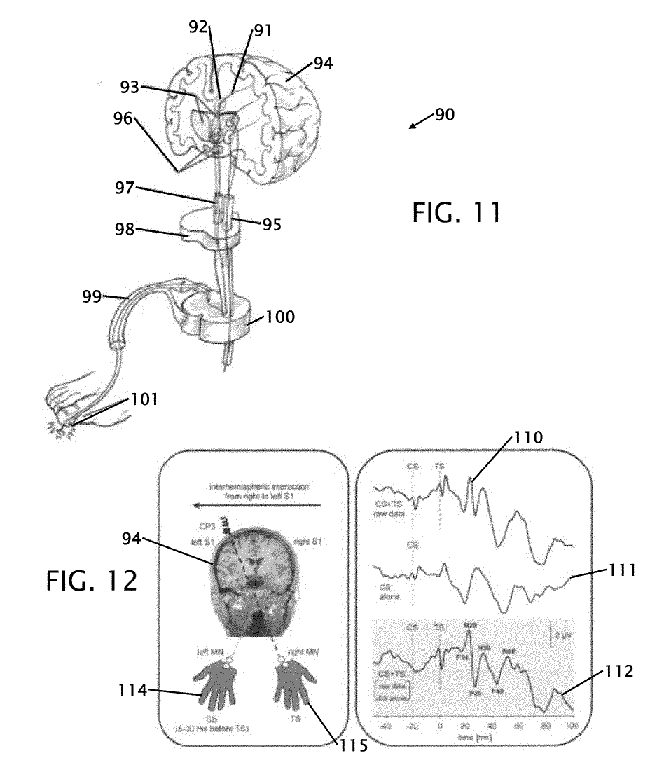

It is contemplated that a conductive plate with a plurality of contacts can be brought in contact with nerves in the remaining limb or spine. The plurality of contacts can be monitored and processed by a computer and then using a microcontroller or VLSI to convert the data from the plurality of contacts to operate mechanical muscles. VLSI circuit programmed to convert analog neurological electrical impulses into a digital protocol for activating a robotic prosthetic. A standardized source code OS is public access as befits the numerous neurorobotic prosthetics and products extending even into cybernetic enhancement, zero-gravity, cosmetic animatronic jewelry, 3D tattoos, entertainment costuming etc. Sensors on the replacement prosthetic can also send signals back through the plurality of contacts to provide feedback to the nervous system to the brain of a person 90. FIG. 11 shows a block diagram of the nervous system detection of pain. This figure shows the pain receptors 101 sends signal through the spinal nerve 99 and into the spinal cord 100. The signal then passes through the brain stem 98 through the neospinothalamic pathway 95 and the paleospinothalamic pathway 97. The hypothalamous 96 transfers the signal to the thalamous 93, where the signal is interpreted by the limbic forebrain to the cerebral cortex in the cerebrum 94.

A static rigid bolted mounted receiver wire thread insert and abutment for a standard prosthetic. An internal/external abutment which allows for surrounding residual muscle to be attached and used to activate mechanical prosthetic appendage. An internal/external abutment containing, a contact plate with sufficient gold contact points to attach residual nerve endings, during implanting surgery with assist from Nerveana by Medivison Ventura, Calif. nerve identification, send, receive, target so as to exploit proprioceptive memory or retraining.

FIG. 12 shows a graphical representation of the nerve sensory detection. In this figure the sensory input from the left hand 114 (CS) and the right hand 115 (TS) is transmitted to the brain 94. The graph shows the raw CS+TS data 110, the CS data alone 111 and the new response 112 to the stimuli as a function of time.

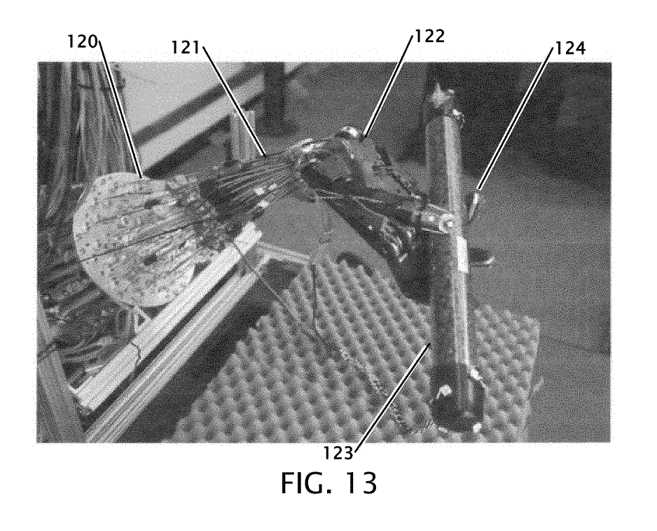

FIG. 13 shows a robotic interface from a sensory and muscle interface. Surgical robots, micro-nano-robotics, soft robotics, industrial robotics, humanoid robotics, neuro-robotics, prosthetics, neural engineering, rehabilitation engineering, bio-inspired robotics, biomedical signal processing, marine robotics, service robotics and ambient assisted living, educational robotics and their ethical, legal, social and economic implications. The BioRobotics Institute is an integrated system aimed at innovation research, education and technology transfer, and it intends to create new companies in high tech sectors.

Soft Robotics which imitate biological tissue and function is capable of said function(s) which rigid structure cannot achieve.

The growing need for robots in service tasks, in unstructured environments, in contact with humans, is leading to release the basic assumption of rigid parts in robotics. The role of soft body parts appears clear in natural organisms, to increase adaptability and robustness. Compliance, or softness, are also needed for implementing the principles of embodied intelligence, or morphological computation, a modern view of intelligence, attributing a stronger role to the physical body and its interaction with the environment. One simple example would imitate and octopus tentacle or tadpole tail.

The “Micro-Nano-Bio Systems and Targeted Therapies” Lab has the mission of studying phenomena at the Mill-, Micro- and nanoscale, to invent new solutions and to engineer processes at such scales, in order to develop advanced technological components and the enable minimally invasive therapies. A high level of interdisciplinary features the group, whose research efforts are at the edge between robotics micro-mechanics, materials science and molecular biology. The limb replacement in this figure has a base control module 122 with a replacement arm 121, wrist 122 and digits 124. This robotic forearm can lift and sense a load 123 to provide the proper grasp force. Sensory inputs on the fingers 124 provide feedback on grip force and can further provide sensory information regarding temperature and surface texture.

There are a number of organizations such as, but not limited to Meridan which is a European Commission through the Seventh Framework Programme that is working with carbon-based biomimetics interfaces for innovative neuroprosthetics. Organizations working in this area with a goal to optimize novel electrode technologies, using nanobiology and cellular physiology, integrated within nanodiamond materials processing towards a new generation of high-resolution chronic implants, with high stability and low biofouling. The application of diamond technology innovated the current bionic devices and provides them with advanced functionalities, better performance and higher market impact towards preclinical stage testing.

Integrated knowledge on nano-materials, electrode design, and electronics are used to design devices operating in vivo, with sensory and motor neural signals for bi-directional biomimetic interfaces. This works towards advanced voluntary control prosthetics and nerve regeneration.

The drive is towards high degrees of freedom prosthetic actuators in man-machine interfaces, using the developed devices in combination with minimally-invasive surgery. The goal is a high-resolution ENG and neuromuscular amplification.

There is further reduced discomfort such as heat, sweating and chafing. Patients experience improved sensory feedback because the phenomenon of sensation through the bone (Osseo perception) is present. This type of integration is adaptable to thumb amputation where no other integration provides the Osseo perception to the hand and wrist.

Thus, specific embodiments of a dental/prosthetic implant have been disclosed. It should be apparent, however, to those skilled in the art that many more modifications besides those described are possible without departing from the inventive concepts herein. The inventive subject matter, therefore, is not to be restricted except in the spirit of the appended claims.

1. A dental implant comprising:
a wire threaded insert;
a tapped hole in a bone;
a dental implant having an abutment;
said abutment having a first end that is threaded and an opposing second end with a tapped hole;
said dental implant further includes a threaded fastener configured to engage in said tapped hole in said abutment, and
said dental implant further a tapered hole.

2. The dental implant according to claim 1 wherein said wire threaded insert being fabricated from titanium.

3. The dental implant according to claim 2 wherein said titanium is nanostructured.

4. The dental implant according to claim 1 further includes a second wire threaded insert.

5. The dental implant according to claim 1 wherein said wire threaded insert is at least partially coiled and threaded into said tapped hole.

6. The dental implant according to claim 5 wherein said wire threaded insert expands with said tapped hole.

7. The dental implant according to claim 1 wherein said abutment is tapered to match said tapered hole in said dental implant.

8. The dental implant according to claim 1 wherein wire in said wire threaded insert is square in cross-section.

9. The dental implant according to claim 1 wherein said wire threaded insert is square or tapers.

10. The dental implant according to claim 1 wherein said abutment is selected from a group consisting of an aesthetic abutment, an angled contoured abutment, a ball attachment abutment, a casting abutment, a full contour abutment, a gold castable abutment, a plastic castable abutment, a global positioning satellite (GPS) abutment, a RFID abutment, an ID with medical records abutment, a miniature transceiver abutment, an angled abutment, a healing abutment, a locator abutment, a lab analogs abutment, a multi-unit abutment, a plastic non-engaging castable abutment, a plastic temporary abutment, a screw receiving abutment, an angled screw receiving abutment, a straight contoured Zirconia or Titanium abutment, a straight snap-on abutment and a transfer abutment.

11. A prosthetic implant comprising:
a wire threaded insert;
a tapped hole in a bone;
said prosthetic implant having an abutment;
said abutment having a first end that is threaded and an opposing second end with a tapped hole;
said prosthetic implant further includes a threaded fastener configured to engage in said tapped hole in said abutment, and
said prosthetic implant further a tapered hole.

12. The prosthetic implant according to claim 11 wherein said wire threaded insert being fabricated from titanium.

13. prosthetic implant according to claim 13 wherein said titanium is nanostructured.

14. The prosthetic implant according to claim 11 further includes a second wire threaded insert.

15. The prosthetic implant according to claim 11 wherein said wire threaded insert is at least partially coiled and threaded into said tapped hole.

16. The prosthetic implant according to claim 15 wherein said wire threaded insert expands with said tapped hole.

17. The prosthetic implant according to claim 11 wherein said abutment is tapered to match said tapered hole in said prosthetic implant.

18. The dental/prosthetic implant according to claim 11 further includes a contact plate having a plurality of contacts configured for contacting nerves.

19. The dental/prosthetic implant according to claim 18 contacting nerves include a group selected from send, receive, muscle control, temperature, fatigue, angle, and touch.

20. The prosthetic implant according to claim 11 wherein said abutment is selected from a group consisting of an aesthetic abutment, an angled contoured abutment, a ball attachment abutment, a casting abutment, a full contour abutment, a gold castable abutment, a plastic castable abutment, a global positioning satellite (GPS) abutment, a RFID abutment, an ID with medical records abutment, a miniature transceiver abutment, an angled abutment, a healing abutment, a locator abutment, a lab analogs abutment, a multi-unit abutment, a plastic non-engaging castable abutment, a plastic temporary abutment, a screw receiving abutment, an angled screw receiving abutment, a straight contoured Zirconia or Titanium abutment, a straight snap-on abutment and a transfer abutment.
